(19) United States
(12) Patent Application Publication
(10) Pub. No.: US 2007/0218110 A1
(43) Pub. Date: Sep. 20, 2007

(54) NOVEL ENHANCED PROCESS FOR IMPARTING THERAPEUTIC AND NUTRITIVE LEVELS OF FLAXSEED TO MAMMALS

(75) Inventor: Darlena Goetz, Long Beach, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: FlaxSnax, Inc.

(21) Appl. No.: 11/687,468

(22) Filed: Mar. 16, 2007

Related U.S. Application Data
(60) Provisional application No. 60/782,802, filed on Mar. 16, 2006.

(51) Int. Cl. A61K 47/00; A61K 36/55

(52) U.S. Cl. 424/439; 424/768

(57) ABSTRACT

A process for imparting healthful dosages of flaxseed to mammals and a muffin-based regimen for providing a healthful and therapeutic dosage of Alpha-Linolenic Acid (ALA), an essential omega-3 fatty acid, in combination with desiderata selected from the group consisting of cinnamon, fiber, bananas, walnuts, carrots—optionally, without any negative impact upon the subject articles' ability to maintain their palatable and gustatorily attractive character.

### BACKGROUND OF THE DISCLOSURE

The present disclosure relates to technologies for using delivery vehicles to impart healthful dosages of flaxseed and other beneficial nutritives to mammals. In particular, the instant disclosure teaches improved ways to encourage humans to consume healthier amounts of flaxseed, more frequently.

In particular, the present disclosure combines palatability with therapeutically effective amounts of flaxseed, in conjunction with other desiderata, effectively creating heretofore unprecedented compliance options for those in need of essential oils, fiber, and a balanced approach to being healthy.

Although heightened awareness impacts the diet and lifestyle of many concerned with both cardiovascular and general aspects of living a healthy lifestyle, in today's active world an ostensive disconnect exists between wishing to manage diet, control caloric intake given day-to-day activities, and to feel energized and able to face daily challenges. The present disclosure addresses and ameliorates these longstanding needs.

The teachings of the present disclosure provide an avenue to obtain redress for certain of these goals, and in so doing provide a useful and valuable tool for those interested in being healthier, without compromising on taste.

### SUMMARY OF THE DISCLOSURE

There are presented novel compositions of matter, formulations, methods and dry-blend mixtures which both present healthy oils and other nutritionally balanced formulations, heretofore unavailable with the technical advancements of the present disclosure.

A process and products for imparting healthful dosages of flaxseed to mammals includes business methods and baked goods-based regimens for providing a healthful and therapeutic dosage of Alpha-Linolenic Acid (ALA), an essential omega-3 fatty acid, in combination with desiderata selected from the group consisting of cinnamon, fiber, bananas, walnuts, carrots, cranberries, and related supplements, complements and other healthful additives,—optionally, without any negative impact upon the subject articles' ability to maintain their palatable and gustatorily attractive character.

Briefly stated, Flax (commonly know to Artisans as linseed or flaxseed) (Linum usitatissimum) and its oil are perhaps the most widely—available, botanical sources of omega-3 fatty acid, according to current literature and the science that is in the public domain. Flaxseed oil consists of ca. 55% ALA (alpha-linolenic acid) The present disclosure is directed to processes and methods for imparting healthful dosages of flaxseed to mammals and likewise includes a business method and baked goods-based regimen for providing a healthful and therapeutic dosage of Alpha-Linolenic Acid (ALA), an essential omega-3 fatty acid, in combination with desiderata selected from the group consisting of cinnamon, fiber, bananas, walnuts, carrots, and cranberries, inter alia—optionally, without any negative impact upon the subject articles' ability to maintain their palatable and gustatorily attractive character.

At a root technical level flaxseed, as used in accordance with the teachings of the present disclosure, contains approximately three times as much omega-3 as omega-6. Prior to the advent of the instant teachings delivery vehicles for the same has been mostly limited to pills and noxious fish-oil basal supplements.

For example, 15 g of flaxseed oil provides ca. 8 g of ALA, which is converted in the body to EPA and then DHA at an efficiency of (5%-10%), and (2%-5%) respectively. In other words, flaxseed oil is among the most efficient sources of ALA, which as becomes clear below—is essential for good health. The instant teaching broadens accessibility for the same to numerous consumers, including special needs and cardio-vascularly challenged people.

Accordingly to the present disclosure, there are provided processes and business methods for imparting therapeutic and nutritive levels of flaxseed to mammals comprising, in combination ascertaining a healthful dosage regiment for a group of related users; procuring freshly milled flaxseed, or a simulacrum or synthetic source of alpha 3 omega fatty acids; formulating at least a recipe permitting the flaxseed to be combined with at least one of cinnamon, fiber, banana-walnut, carrot, and pumpkin; producing a dry-blend mix effective for being further heat-processed into edible an tasty end products; and creating finished products having a positive tasting element effective to induce users to undertake ongoing ingestion of the same; and repeating optionally any of the previous or desired steps to make the finished products and their nutritive and therapeutical benefits available and known to consumers.

According to a feature of the present disclosure, there is provided a process and business method for procuring an organically generated source of those essential oils known as ALA.

According to another feature of the present disclosure, there is provided a product produced by adding at least about 1.1 to 3.3 tablespoons of flaxseed.

According to yet another feature of the current disclosure, there is provided a process wherein the producing step further comprises generating a scalable and quality controlled batch mixture which may be heat-processed by conventioned or novel means whereby end-products may be at least one of frozen, refrigerated, micro-wave processed and still maintain their palatable and gustatorily attractive character.

According to yet a still further feature of the current disclosure, there is provided a product further comprising a method for targeting at least one disease state selected from the group consisting of cardiovascular disease, ophthalmic disease states, respiratory ailments, obesity, diabetes, addiction, menopause, neurological challenges, inflammatory joint ailments, auto-immune disease states, malnutrition, dermatological challenges, attention deficit disorder, gastro-intestinal or colonic disease, pms, depression, hair-thinning and any other ailment covered by health insurance or recognized by providers of healthcare services to need to be addressed in certain groups of people.

According to another feature of the instant disclosure, the product further comprises at least one of a baked good selected from the group of cookies, loaves, bread products, cupcakes, cakes, pannini, breakfast and/or traditional muffins, pancakes, dinner muffins, and the like articles of food.

According to another feature, the product in one embodiment further comprises at least about 3 tablespoons of flaxseed in a conventional (about 4.4 oz.) muffin, for example, those skilled in the art readily understand that this means the flaxseed level can be varied driven by the gross size of the baking article and the particular application for which the same is tailored, as becomes clear below within the description and as defined by the claims appended hereto.

### DETAILED DESCRIPTION OF THE PRESENT DISCLOSURE

The present inventor has discovered that Omega-3 fatty acids, optionally—in combination with other ingredients (as needed, desired, or otherwise indicated), can be supplied in good tasting foods, for example muffins, by a process for imparting therapeutic and nutritive levels of flaxseed to mammals. Cookies, muffins and well accepted baked goods under the teachings of this disclosure are currently available from FLAXSNAX® of Long Beach, Calif. 90803.

Omega-3 fatty acids are polyunsaturated fatty acids found in oil from oily fish and vegetable sources such as the seeds of chia, perilla, flax, purslane, lingonberry and hemp. Omega-3 fatty acids are classified as essential because they cannot be synthesized in the body; they must be obtained from food. Important omega-3 fatty acids in human nutrition are: ALA (α-linolenic acid), EPA (eicosapentaenoic acid), and DHA (docosahexaenoic acid).

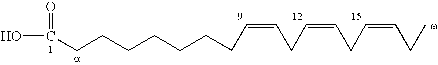

Chemical structure of Alpha-Linolenic Acid (ALA), an essential omega-3 fatty acid, found in flaxseed.

The term “omega-3” (aka “n−3”, “ω-3”) signifies that the first double bond exists as the third carbon—carbon bond from the terminal methyl end (ω) of the carbon chain. Omega-3 fatty acids which are important in human nutrition are: Alpha-Linolenic Acid (18:3, ALA), Eicosapentaenoic Acid (20:5, EPA), and Docosahexaenoic Acid (22:6, DHA). These three polyunsaturates have either 3, 5 or 6 double bonds in a carbon chain of 18, 20 or 22 carbon atoms, respectively. All double bonds are in the cis-configuration, i.e. the two hydrogen atoms are on the same side of the double bond.

Structurally, Omega-3 fatty acids are helically twisted, because every cis- double bond, separated by a methylene group, changes the carbon chain's direction. This configuration may explain a host of biological phenomena observed in structures that are rich in polyunsaturated fatty acids, especially the lipid bilayer of the cell membrane.

The seminal 1992 paper by biochemist William E. M. Lands provides an overview of the research into omega-3 fatty acids, in historical context, where the essential nature of said oil was set forth, or at least pre-viewed based upon the structure and how that morphology likely works within the context of our physiology. Until now, most consumers have not been able to readily understand or appreciate this.

The ‘essential’ fatty acids were given their name when researchers found that they were essential to normal growth in young children and animals. (Note that they modern definition of ‘essential’ can be considered to be considerably more stringent.) A small amount of omega-3 in the diet (˜1% of total calories) enabled normal growth, and increasing the amount had little to no additional benefit, in empirical studies done with children.

Likewise, researchers found that omega-6 fatty acids (such as γ(gamma)-linolenic acid and arachidonic acid) play a similar role in normal growth. However, they also found that omega-6 is “better” at supporting dermal integrity, renal function, and parturition. This led researchers to concentrate study on omega-6, and it is only in recent decades that omega-3 has become of interest. The ratio turns out to be key for mammals to process these essentials.

In 1963 it was discovered that the omega-6 arachiodonic acid is converted by the body into pro-inflammatory agents called prostaglandins. By 1979 more of what are now known as eicosanoids were discovered: thromboxanes, prostacyclins and the leukotrienes. The eicosanoids, which have important biological functions, typically have a short active lifetime in the body, starting with synthesis from fatty acids and ending with metabolism by enzymes. However if the rate of synthesis exceeds the rate of metabolism, the excess eicosanoids may have deleterious effects. Researchers found that omega-3 is also converted into eicosanoids, but at a much slower rate. If both omega-3 and omega-6 are present, they will “compete” to be transformed, so the amount of omega-3 present is directly related to a decrease in the rate of eicosanoid production.

This competition was recognized as important when it was found that thromboxane is a factor in the clumping of platelets, which leads to thrombosis. The leukotrienes were similarly found to be important in immune/inflammatory-system response, and therefore relevant to arthritis, lupus, and asthma. These discoveries led to greater interest in finding ways to control the synthesis of omega-6 eicosanoids; one way being, of course, the consumption of greater amounts of omega-3.

On Sep. 8, 2004, the U.S. Food and Drug Administration gave “qualified health claim” status to eicosapentaenoic acid (EPA) and docosahexaenoic acid (DHA) omega-3 fatty acids, stating that “supportive but not conclusive research shows that consumption of EPA and DHA omega-3 fatty acids may reduce the risk of coronary heart disease.”

In fact, omega-3 fatty acids [specifically the long carbon chain eicosapentanoic acid (EPA) and docosahexanoic acid (DHA)] have been found to be so potentially beneficial to humans that fish oil supplements are slowly gaining some recognition as a probable key element in improving human health in general. Unlike the present invention, a significant potential risk is the possibility of vitamin poisoning from taking large doses of supplements which contain large quantities of vitamins (particularly vitamin A) in addition to omega-3 fatty acids.

Perhaps the greatest risk of fish oil omega-3 supplementation is heavy metal poisoning by the body's accumulation of traces of heavy metals, in particular; mercury, lead, nickel, arsenic and cadmium and other contaminants (PCBs, furans, dioxins), which may be found in less refined fish oil supplements.

Likewise, omega-3's are definitively known to have membrane-enhancing capabilities in brain cells that facilitate necessarily positive results. One medical explanation is that omega-3's play a role in the fortification of the myelin sheaths. Not coincidentally, omega-3 fatty acids comprise approximately eight percent of the average human brain according to the late Dr. David Horrobin, a pioneer in fatty acid research. Ralph Holman of the University of Minnesota, another major researcher in studying essential fatty acids, surmised how omega-3 components are analogous to the human brain by stating that “DHA is structure, EPA is function.”

Consequently, the past decade of omega-3 fatty acid research has procured some Western interest in omega-3's as being a legitimate ‘brain food.’ Still, recent claims that intelligence quota and verbal reasoning skills are increased on account of omega-3's consumed by pregnant mothers remain unreliable and controversial. An even more significant locus of research, however, lies in the role of omega-3's as a non-prescription treatment for certain psychiatric and mental diagnoses and has become a topic of much research and speculation. This is particularly true since the downsides are minimal to non-existent, as according to the instant disclosure.

Dr. Andrew Stoll and his colleagues at Harvard University were among the first to accomplish the testing of such hypothetical research through a 1999 double-blind placebo study done with thirty patients having been diagnosed with manic depression. This experiment was designed for nine months. He rendered his results by affording olive oil capsules to fifteen placebo control subjects and nine grams of pharmaceutical-quality EPA and DHA supplements to the fifteen others. In doing so he was able to make the general distinction between the placebo group failing to improve while the Omega-3 group experienced a noticeable degree of recovery. Though Stoll believes that the 1999 experiment was not as optimal as it could have been and has accordingly pursued further research, the foundation has been laid for more researchers to explore the theoretical association between absorbed omega-3's and signal transduction inhibition in the brain.

Should enough research that is currently underway come to confirm the legitimacy of this association, then a debate and reassessment will of course be necessitated between Omega-3's and such over-the-counter prescription bipolar treatments as Eskalith®, or branded versions of Lithium Carbonate. Some physicians and psychiatric specialists in the United States do allow willing bipolar patients to use Omega-3 supplements as conditional treatments. Omega-3's, unlike Eskalith, are less expensive and do not commonly induce such side effects as diarrhea, drowsiness, and fatigue. More time is needed, however, for Omega-3-induced signal transduction inhibition in the brain to become a thoroughly proven association. Until then, the present application of what some may consider speculation remains verifiable.

Although fish is a dietary source of omega-3 fatty acids, fish do not synthesize them, they obtain them from the algae in their diet. For this reason, there is often a significant difference in EPA and DHA concentrations in farmed vs wild caught fish, with the latter having better access to algal sources (but also both sources have been implicated in mercury, PCB, PBBE and other noxious contaminant level studies). In point of fact, botanical sources are more compelling, as illustrated by the attached table, Flaxseed has 55% as discussed above, and the next three are:

TABLE 1

Purslane (aka portulaca) (Portulaca oleracea) - approx. 47%
Lingonberry (aka cowberry) (Vaccinium vitis-idaea) - approx. 44%
Hemp (aka cannabis) (Cannabis sativa) - approx. 20%

Similar debates exist about other sources, and how they are assimilated by mammals. By way of further example, A P Simopoulos found that chickens which wandered freely in the countryside, eating rich sources of omega-3 such as purslane, produced eggs with 20 times more omega-3 than eggs of grain-fed chickens. This was significant and has led to further improvements, studies and other focuses supportive of the instant teachings.

In the early 21st century, improving the ALA content of feed provided to egg laying hens have increased the DHA concentration of the resulting eggs. Unlike traditional eggs which are very high in pro-inflammatory omega-6, the increased DHA eggs produce a healthier omega-6 to omega-3 ratio.

Meat from grass-fed animals is often higher in omega-3 than meat from the corresponding grain-fed animal. The n6:n3 ratio of grass-fed beef is about 2:1, making it a more useful source of omega-3 than grain-fed beef (ca, 4:1). Commercially available lamb is almost always grass-fed, and subsequently higher is omega-3 than other common meat sources.

Milk and cheese from grass-fed cows may be good sources of omega-3. Milk from non-organic, grain-fed cows may contain about two-thirds less omega-3. One UK study showed that half a pint of milk provides 10% of the recommended daily intake (RDI) of ALA, while a “matchbox sized piece of organic cheese will give you up to 88%.” The issue here is that there are serious downsides to each of said alternate sources, in contradistinction to the teachings of the present invention.

By way of yet still further example, the microalgae Crypthecodinium cohnii and Schizochytrium are rich sources of DHA (22:6 ω-3), produced commercially in bioreactors. Oil from brown algae (kelp) is a source of EPA.

### Cardiovascular Medicine Clearly Shows Flaxseed is Key

Clinical Studies indicate that the ingested ratio of omega 6 to omega 3 (especially Linoleic vs Alpha Linolenic) fatty acids may be important to maintaining cardiovascular health, and ingesting the same regularly is a key to getting full benefits from it.

Both omega-3 and omega-6 fatty acids are essential, i.e., humans must consume them in the diet. Omega-3 and omega-6 compete for the same metabolic enzymes, thus the omega-6:omega-3 ratio will significantly influence the ratio of the ensuing metabolites, (e.g., prostaglandins, leukotrienes, eicosanoids etc.), and will alter the body's metabolic function. Metabolites of omega-6 are significantly more inflammatory (esp, arachidonic acid) than those of omega-3. This necessitates that omega-3 and omega-6 be consumed in a balanced proportion: the ideal ratio of omega-6:omega-3 being from 1:1 to 5:1. Studies suggest that the evolutionary human diet, rich in seafood, nuts and other sources of omega-3, may have provided a ratio of close to 1:1.

As of March 2006, the FDA has not issued an official RDA for omega-3 or omega-6 fatty acids. Simopoulos, et al recommend daily intakes of three omega-3 forms; 650 mg of EPA and DHA, and 2.22 g of ALA, and one omega-6 form: 4.44 g of LA. This translates to a 3:2 omega-6 to omega-3 ratio. (i.e. 1.5:1).

Unfortunately, “typical” Western diets provide ratios of between 10:1 and 30:1—i.e., dramatically skewed toward omega-6. This is due to the abundance of omega-6 oils such as soy, sunflower, cottonseed, canola, peanut and corn oil. These oils are much better suited for frying and longer storage than are the less stable omega-3 oils, thus western taste for fried and processed foods increases omega-6 consumption. There is likely no compelling reason that a healthy person consuming a typical western diet should even need to supplement their diet with omega-6.

Chia and Flax both provide very good omega-6:omega-3 ratios of approximately 1:3 (skewed toward omega-3), and are therefore capable of significantly improving the omega-6:omega-3 ratio in the traditional western diet. These sources contain omega-3 in Alpha-Linolenic Acid (ALA) form which competes with omega-6 for the delta-6 desaturase enzyme (rate-limiting step), thus may help prevent an excess of the omega-6 metabolyte arachidonic acid (AA) in the body.

Contrary to much conventional wisdom, some foods touted to be high in omega-3 are even higher in omega-6. Although these may be beneficial choices for maintaining a healthy ratio, they may not be relied upon therapeutically to achieve the ideal omega-6 to omega-3 ratio from the typical western diet.

Coconut oil and palm oil contain high levels of stearic acid a saturated fatty acid, and have very little omega-6 or omega-3. These are tolerant of high temperatures, and may be a good choice for general-purpose cooking, but the saturated fat content may make them unsuitable for some consumers. The oil from the common European Walnut (Juglans regia) contains roughly 5:1 omega-6:omega-3 ratio, making Walnuts acceptable, yet unable to improve the overall omega-3Lomega-6 ratio of a typical western diet, Black walnuts (Juglans nigra) have a less favorable 11:1 omega-3:omega-6 ratio.

Grass-fed beef may contain a more beneficial ratio of omega-6 to omega-3 than grain-fed beef. Although the difference is not dramatic, it is measured by one university study as roughly 2:1 vs. 4:1, respectively.

What is unexpectedly better than dictated by empirical data are the excellent results of combining, for example, flaxseed, cinnamon and fiber. In order to overcome the ratios and be compliant, people are turning to healthy sources that taste good. The present disclosures does this.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A process for imparting therapeutic and nutritive levels of flaxseed to mammals comprising, in combination:
ascertaining a healthful dosage regimen for at least a subject;
procuring flaxseed, as a source of alpha 3 omega fatty acids;
formulating at least a recipe permitting the flaxseed to be combined optionally with at least one of cinnamon, fiber, banana-walnut, carrot, cranberry, pumpkin and the like desiderata;
producing at least one of a dry-blend mix, a frozen ready-made formulation, a SCOOP-N-BAKE® type of admixture, and other pre-baked packetized aliquot of mixture, which is effective for being further heat-processed into edible and tasty end products; and
creating finished products having a positive tasting element effective to induce users to undertake ongoing ingestion of the same; and
repeating optionally any of the previous or desired steps to make the finished products and their nutritive and optionally, at the preference of the vendor, or by license agreement, the intrinsic therapeutic benefits available and known to consumers, as desired or not.

2. The process of claim 1, said procuring step further comprising an organically generated source of the same.

3. The process of claim 2, said formulating step further comprising adding at least about a tablespoon to 3.8 tablespoons of flaxseed, depending on the article size of the subject baked good.

4. A product, produced by the process of claim 3.

5. The process of claim 1, the producing further comprises:
generating a scalable and quality controlled batch mixture which may be heat-processed by conventional or novel means whereby end-products may be at least one of refrigerated, frozen, baked, toasted, convection heated, micro-wave processed, or otherwise finished and still maintain their palatable and gustatorily attractive character.

6. A product, produced by the process of claim 5.

7. The process of claim 1, the ascertaining step further comprising targeting at least one disease state selected from the group consisting of cardiovascular disease, ophthalmic disease states, respiratory ailments, obesity, diabetes, addiction, menopause, neurological challenges, inflammatory joint ailments, auto-immune disease states, malnutrition, dermatological challenges, attention deficit disorder, gastro-intestinal or colonic disease pms, depression, hair-thinning and other ailments covered by health insurance and recognized by providers of healthcare services to need to be addressed in mammals.

8. A product, produced by the process of claim 7.

9. The product of claim 4, further comprising at least one baked good such as a breakfast or traditional cookie, muffin, bread, roll, pannini, loaf, pancake, dinner muffin and the like consumable which may be heated.

10. The product of claim 4, wherein said baked good contains at least about 3 tablespoons of flaxseed, for a conventional article, or a scaled down ratio proportional to the size.

11. A product, produced by the process of claim 1, further comprising at least about 0.3 tablespoons of flax.

12. A product, produced by the process of claim 1, further comprising at least about 0.5 tablespoons of flax.

13. A product, produced by the process of claim 1, further comprising at least about 0.7 tablespoons of flax.

14. A product, produced by the process of claim 1, further comprising at least about 0.9 tablespoons of flax.

15. A product, produced by the process of claim 1, further comprising at least about 1.1 tablespoons of flax.

16. A product, produced by the process of claim 1, further comprising at least about 1.5 to 2 tablespoons of flax.

17. A product, produced by the process of claim 1, further comprising at least about 2 to 2.5 tablespoons of flax.

18. A product, produced by the process of claim 1, further comprising at least about 2.5 to 2.7 tablespoons of flax.

19. A product, produced by the process of claim 1, further comprising at least about 2.7 to 3.3 tablespoons of flax.

20. A product, produced by the process of claim 1, further comprising at least about 3 to approximately 3.8 tablespoons of flax, adjustable upward as needed for the particular article in question.
